(19) United States
(12) Patent Application Publication
(10) Pub. No.: US 2022/0331220 A1
(43) Pub. Date: Oct. 20, 2022

(54) PREMIX AND COMPOSITION AND METHODS OF PREPARING THE SAME

(71) Applicant: Conopco, Inc., d/b/a UNILEVER, Englewood Cliffs, NJ (US)

(72) Inventors: Chen QIAN, Shanghai (CN); Jinlong WANG, Shanghai (CN); Liansong XIE, Shanghai (CN)

(21) Appl. No.: 17/642,595

(22) PCT Filed: Sep. 11, 2020
(86) PCT No.: PCT/EP2020/075435

§ 371 Date: Mar. 11, 2022

(30) Foreign Application Priority Data
Sep. 17, 2019 (CN) PCT/CN2019/106212
Nov. 4, 2019 (EP) 19206801.3

(51) Int. Cl. A61K 8/49; A61Q 5/00

(52) U.S. Cl. CPC A61K 8/4926; A61Q 5/006; A61K 2800/882

(57) ABSTRACT

Disclosed is a premix comprising piroctone olamine, a co-solvent selected from sodium salicylate, sodium benzoate, para aminsobenzoic, sodium xylene sulfonate, iso-octanol, hexanediol, phenoxyethanol, carbamide, acetamide, carbamate and mixtures thereof, and water, wherein the weight ratio of co-solvent to water ranges from 1:20 to 1:1. Hair care compositions comprising the premix and methods of preparing the premix and the hair care compositions.

### TECHNICAL FIELD OF THE INVENTION

The present invention relates to a premix comprising piroctone olamine, a co-solvent and water. Moreover, the present invention also relates to a hair care composition comprising the premix and methods of preparing the premix and the hair care composition.

### BACKGROUND OF THE INVENTION

Dandruff control is an important aspect of hair cleansing and care. Anti-dandruff benefit has been provided through hair care compositions including shampoos and hair conditioners. Dandruff is an issue that affects many people globally. The condition is manifested by the shedding of clumps of dead skin cells from the scalp. These are white in colour and provide an aesthetically displeasing appearance. A factor that contributes to dandruff is certain members of the Malassezia yeasts. To combat these, hair care products have been developed including various anti-dandruff agents, for example, piroctone olamine (Octopirox®).

Piroctone olamine is a solid at standard temperature and pressure. It is usually soluble in surfactants of the cleansing phase comprised in a hair care composition. In a traditional production process of hair products, a problem associated with piroctone olamine is that it cannot be dosed directly into a main tank containing surfactants as it tends to attach to the interior wall of the main tank causing cleaning and corrosion concerns.

Efforts have been made to address the problem. One approach is to dissolve piroctone olamine in a premix containing oil such as perfume before adding it to the main tank. However, high shear mixers and cooling jackets are mandatory to ensure efficient dissolution of piroctone olamine in such a premix. It is therefore not an approach favoured by the industry.

The present inventors have now found that piroctone olamine can be efficiently dissolved in a premix containing water and a co-solvent. It has been found unexpectedly that co-solvent such as sodium salicylate, sodium benzoate, para aminobenzoic, sodium xylene sulfonate, iso-octanol, hexanediol, phenoxyethanol, carbamide, acetamide or carbamate can greatly improve the dissolution of piroctone olamine in water in terms of dissolution rate and dosage. The increased dissolution rate and dosage of piroctone olamine provides enhanced anti-dandruff effectiveness and enhances the efficiency of the production process of hair products and even helps save costs.

### SUMMARY OF THE INVENTION

In a first aspect, the present invention is directed to a premix comprising:
(a) piroctone olamine;
(b) a co-solvent selected from sodium salicylate, sodium benzoate, para aminobenzoic, sodium xylene sulfonate, iso-octanol, hexanediol, phenoxyethanol, carbamide, acetamide, carbamate and mixtures thereof; and
(c) water;
   wherein the weight ratio of co-solvent to water (b:c) ranges from 1:20 to 1:1.

In a second aspect, the present invention is directed to a hair care composition comprising the premix of any embodiment of the first aspect of this invention.

In a third aspect, the present invention is directed to a method of preparing a premix of any embodiment of the first aspect of this invention comprising the steps of:
(i) dissolving the co-solvent in water to produce a solution; and
(ii) adding piroctone olamine in a solution of step (i) to obtain the premix.

In a fourth aspect, the present invention is directed to a method of preparing a hair care composition of any embodiment of the second aspect of this invention comprising the steps of:
(i) dissolving the co-solvent in water to produce a solution;
(ii) adding piroctone olamine in the solution of step (i) to obtain a premix;
(iii) mixing the premix of step (ii) with other ingredients to form the hair care composition.

In a fifth aspect, the present invention is directed to a method of treating and/or preventing dandruff comprising the step of applying the hair care composition of any embodiment of the second aspect of this invention onto hair and/or scalp of an individual. The method is preferably non-therapeutic.

All other aspects of the present invention will more readily become apparent upon considering the detailed description and examples which follow.

### DETAILED DESCRIPTION

Except in the examples, or where otherwise explicitly indicated, all numbers in this description indicating amounts of material or conditions of reaction, physical properties of materials and/or use may optionally be understood as modified by the word “about”.

All amounts are by weight of the final hair care composition, unless otherwise specified. It should be noted that in specifying any ranges of values, any particular upper value can be associated with any particular lower value.

For the avoidance of doubt, the word “comprising” is intended to mean “including” but not necessarily “consisting of” or “composed of”. In other words, the listed steps or options need not be exhaustive.

The disclosure of the invention as found herein is to be considered to cover all embodiments as found in the claims as being multiply dependent upon each other irrespective of the fact that claims may be found without multiple dependency or redundancy.

Where a feature is disclosed with respect to a particular aspect of the invention (for example a composition of the invention), such disclosure is also to be considered to apply to any other aspect of the invention (for example a method of the invention) mutatis mutandis.

“Premix”, as used herein, refers to a mixture formed separately and prior to the formulation of final composition.

“Hair care composition”, as used herein, is meant to include a composition for topical application to hair and/or scalp of mammals, especially humans. Such a composition may be generally classified as leave-on or rinse off, and includes any product applied to a human body for also improving appearance, cleansing, odor control or general aesthetics. The composition of the present invention can be in the form of a liquid, lotion, cream, foam, scrub, gel, or bar. Non-limiting examples of such compositions include leave-on hair lotions, creams, and rinse-off shampoos, conditioners, shower gels, or toilet bar. The composition of the present invention is preferably a rinse-off composition, especially preferred being a shampoo or a conditioner and most preferably a shampoo.

Piroctone olamine, also known as Octopirox® and piroctone ethanolamine, is the ethanolamine salt of the hydroxamic acid derivative piroctone. Piroctone olamine has a structure defined by formula (I):

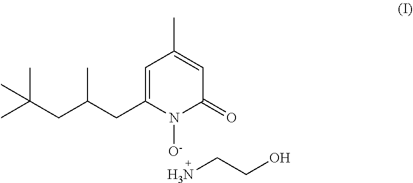

Piroctone olamine is an anti-dandruff active used in the treatment of fungal infections. It helps destroy the fungal infection that is responsible for the dandruff and works against the formation of new dandruff.

Typically, the piroctone olamine is present in an amount of from 0.01 to 70% by weight of the premix, more preferably from 0.1 to 50%, and most preferably from 1 to 30%, based on total weight of the premix and including all ranges subsumed therein.

The co-solvent suitable for use in the premix of the present invention is selected from sodium salicylate, sodium benzoate, para aminobenzoic, sodium xylene sulfonate, iso-octanol, hexanediol, phenoxyethanol, carbamide, acetamide, carbamate and mixtures thereof. It is preferable that the co-solvent is sodium salicylate, sodium benzoate, hexanediol, phenoxyethanol or mixtures thereof, more preferably the co-solvent is sodium salicylate, phenoxyethanol or mixtures thereof. In a particularly preferred embodiment, the co-solvent is a mixture of sodium salicylate and phenoxyethanol.

The co-solvent is employed in the premix to improve the dissolution of piroctone olamine in water. Without wishing to be bound by theory the present inventors believe that the co-solvent may behave as hydrotropic agent which reacts with piroctone olamine to form water-soluble intermolecular complexes. Therefore, the co-solvent is able to increase the solubility of piroctone olamine in water dramatically to form a homogeneous solution.

The co-solvent is preferably present in an amount of from 3 to 70% by weight of the premix, more preferably from 7 to 50%, and most preferably from 10 to 30%, based on total weight of the premix and including all ranges subsumed therein.

Water is the solvent used in the premix. Preferably, the premix comprises water in an amount of from 10 to 90% by weight of the premix, more preferably from 20 to 80% and most preferably from 30 to 70%, based on total weight of the premix and including all ranges subsumed therein.

The premix comprises the co-solvent and water in a weight ratio of from 1:20 to 1:1, preferably from 1:10 to 1:1, more preferably still from 1:6 to 1:1 and most preferably from 1:5 to 1:3, including all ratios subsumed therein.

A hair care composition comprising the premix as described above is also included in the present invention.

It is preferred if the hair care composition of the present invention does not comprise piroctone olamine and/or co-solvent in addition to those included in the premix. When the premix is employed in a hair care composition, the piroctone olamine is typically present in an amount of from 0.01 to 10% by weight of the composition, more preferably from 0.01 to 6%, even more preferably from 0.05 to 2% and most preferably from 0.05 to 1.5%, based on total weight of the hair care composition and including all ranges subsumed therein. The co-solvent is preferably present in an amount of from 0.01 to 4% by weight of the composition, more preferably from 0.1 to 2%, and most preferably from 0.1 to 1%, based on total weight of the hair care composition and including all ranges subsumed therein.

The hair care composition may further comprise a cationic polymer. Suitable cationic polymers may be homopolymers or be formed from two or more types of monomers. The molecular weight of the polymer will generally be between 5,000 and 10,000,000 g/mol, typically at least 10,000 g/mol and preferably from 100,000 to 2,000,000 g/mol. The polymers will have cationic nitrogen containing groups such as quaternary ammonium or protonated amino groups, or a mixture thereof. The cationic nitrogen containing group will generally be present as a substituent on a fraction of the total monomer units of the cationic polymer. Thus, when the polymer is not a homopolymer it can contain spacer non-cationic monomer units. The ratio of the cationic to non-cationic monomer units is selected to give a polymer having a cationic charge density in the required range.

Suitable cationic polymers include, for example, copolymers of vinyl monomers having cationic amine or quaternary ammonium functionalities with water soluble spacer monomers such as (meth)acrylamide, alkyl and dialkyl (meth) acrylamides, alkyl (meth)acrylate, vinyl caprolactone and vinyl pyrrolidine. The alkyl and dialkyl substituted monomers preferably have C1-C7 alkyl groups, more preferably C1-C3 alkyl groups. Other suitable spacers include vinyl esters, vinyl alcohol, maleic anhydride, propylene glycol and ethylene glycol.

Preferably, the cationic polymer is a cationic polysaccharide polymer such as cationic cellulose derivatives, cationic starch derivatives, and cationic guar gum derivatives. Suitably, such cationic polysaccharide polymers have a molecular weight of from 100,000 g/mol to 2,300,000 g/mol, more preferably from 150,000 g/mol to 2,000,000 g/mol. Such cationic polysaccharide polymers preferably have a cationic charge density from 0.1 to 4 meq/g.

Cationic polysaccharide polymers suitable for use in compositions of this invention include those represented by the general formula:

A-O—[R¹—N⁺(R²)(R³)(R⁴)X⁻]

wherein: A is an anhydroglucose residual group, such as a starch or cellulose anhydroglucose residual. R¹ is an alkylene, oxyalkylene, polyoxyalkylene, or hydroxyalkylene group, or combination thereof. R², R³ and R⁴ independently represent alkyl, aryl, alkylaryl, arylalkyl, alkoxyalkyl, or alkoxyaryl groups, each group containing up to about 18 carbon atoms. The total number of carbon atoms for each cationic moiety (i.e., the sum of carbon atoms in R², R³ and R⁴) is preferably about 20 or less, and X is an anionic counterion.

Cationic cellulose is available from Amerchol Corp. (Edison, N.J., USA) in their Polymer JR (trade mark) and LR (trade mark) series of polymers, as salts of hydroxyethyl cellulose reacted with trimethyl ammonium substituted epoxide, referred to in the industry (CTFA) as Polyquaternium 10. Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from Amerchol Corp. (Edison, N.J., USA) under the tradename Polymer LM-200.

Other suitable cationic polysaccharide polymers include quaternary nitrogen-containing cellulose ethers (e.g. as described in U.S. Pat. No. 3,962,418) and copolymers of etherified cellulose and starch (e.g. as described in U.S. Pat. No. 3,958,581).

A particularly preferred type of cationic polysaccharide polymer that can be used in compositions of the present invention is a cationic guar gum derivative, such as guar hydroxypropyltrimonium chloride (for example, commercially available from Solvay in their Jaguar trademark series or from Ashland in their N-Hance trademark series). Examples of such materials are Jaguar® C-13S, Jaguar® C-14S, Jaguar® C-17, Jaguar® Excel, Jaguar® C-162, Jaguar® C-500, Jaguar® Optima, Jaguar® LS, N-Hance™ BF17, N-Hance™ BF13 and N-Hance™ CCG45.

Mixtures of any of the above cationic polymers may be used. The cationic polymer preferably comprises cationic cellulose, cationic guar or mixtures thereof. Guar hydroxypropyltrimonium chloride is particularly preferred.

When used, the cationic polymer will generally be present in the hair care composition of the present invention in an amount of from 0.001 to 1% by weight of the hair care composition, more preferably from 0.01 to 0.5%, and most preferably from 0.03 to 0.3%, based on total weight of the hair care composition and including all ranges subsumed therein.

The pH of the composition is preferably equal to or higher than 4.0, more preferably in the range of 4.0 to 7.0.

It is preferable that the hair care composition also comprises a conditioning agent to provide conditioning benefit. Preferably, the hair care composition comprises discrete dispersed droplets of a water-insoluble conditioning agent, which has a mean droplet diameter (D_{3,2}) of less than 15 microns, preferably less than 10 microns, more preferably less than 5 microns, most preferably less than 3 microns. The mean droplet diameter (D_{3,2}) of a water-insoluble conditioning agent may be measured by means of a laser light scattering technique, for example using a 2600D Particle Sizer from Malvern Instruments. The water-insoluble conditioning agent may include non-silicone conditioning agent comprising non-silicone oily or fatty materials such as hydrocarbon oils, fatty esters and mixtures thereof. Preferably, the water-insoluble conditioning agent is emulsified silicone oil.

Suitable silicones include polydiorganosiloxanes, in particular polydimethylsiloxanes which have the CTFA designation dimethicone. Also suitable for use in compositions of this invention (particularly shampoos and conditioners) are polydimethyl siloxanes having hydroxyl end groups, which have the CTFA designation dimethiconol. Also suitable for use in compositions of this invention are silicone gums having a slight degree of cross-linking, as are described for example in WO 96/31188. Preferably, the silicone oil comprises dimethicone, dimethiconol or a mixture thereof.

Suitable emulsified silicones for use in the hair care compositions of this invention are available as pre-formed silicone emulsions from suppliers of silicones such as Dow Corning and GE silicones. The use of such pre-formed silicone emulsion is preferred for ease of processing and control of silicone particle size. Such pre-formed silicone emulsions will typically additionally comprise a suitable emulsifier, and may be prepared by a chemical emulsification process such as emulsion polymerisation, or by mechanical emulsification using a high shear mixer. Examples of suitable pre-formed silicone emulsions include DC1785, DC1788, DC7128, all available from Dow Corning. These are emulsions of dimethiconol/dimethicone.

Another class of silicones which may be used are functionalized silicones such as amino functional silicones, meaning a silicone containing at least one primary, secondary or tertiary amine group, or a quaternary ammonium group. Examples of suitable amino functional silicones include polysiloxanes having the CTFA designation “amodimethicone.”

The conditioning agent is generally present in hair care composition of this invention in an amount from 0.05 to 15%, preferably from 0.1 to 10%, more preferably from 0.5 to 8%, most preferably from 1 to 5%, based on total weight of the hair care composition and including all ranges subsumed therein.

In a preferred embodiment, the hair care composition is a shampoo. Thus in a preferred embodiment the composition comprises a cleansing surfactant. Non-limiting examples of suitable anionic cleansing surfactants are alkyl sulphates, alkyl ether sulphates, alkaryl sulphonates, alkanoyl isethionates, alkyl succinates, alkyl sulphosuccinates, alkyl ether sulphosuccinates, N-alkyl sarcosinates, alkyl phosphates, alkyl ether phosphates, and alkyl ether carboxylic acids and salts thereof, especially their sodium, magnesium, ammonium and mono-, di- and triethanolamine salts. The alkyl and acyl groups generally contain from 8 to 18, preferably from 10 to 16 carbon atoms and may be unsaturated. The alkyl ether sulphates, alkyl ether sulphosuccinates, alkyl ether phosphates and alkyl ether carboxylic acids and salts thereof may contain from 1 to 20 ethylene oxide or propylene oxide units per molecule. Typical anionic cleansing surfactants for use in compositions of the invention include, but not limited to, sodium oleyl succinate, ammonium lauryl sulphosuccinate, sodium lauryl sulphate, sodium lauryl ether sulphate, sodium lauryl ether sulphosuccinate, ammonium lauryl sulphate, sodium dodecylbenzene sulphonate, triethanolamine dodecylbenzene sulphonate, sodium cocoyl isethionate, sodium lauryl isethionate, lauryl ether carboxylic acid, sodium N-lauryl sarcosinate or mixtures thereof. Preferred anionic cleansing surfactants are the alkyl sulphates and alkyl ether sulphates. Preferred alkyl sulphates are C₈₋₁₈ alky sulphates, more preferably C₁₂₋₁₈ alkyl sulphates, preferably in the form of a salt with a solubilising cation such as sodium, potassium, ammonium or substituted ammonium. Examples are sodium lauryl sulphate (SLS) or sodium dodecyl sulphate (SDS). It is particularly preferred that the anionic cleansing surfactant is alkyl ether sulphate. Preferred alkyl ether sulphates are those having the formula: RO(CH₂CH₂O)ₙSO₃M; wherein R is an alkyl or alkenyl having from 8 to 18 (preferably 12 to 18) carbon atoms; n is a number having an average value of greater than at least 0.5, preferably between 1 and 3, more preferably between 2 and 3; and M is a solubilising cation such as sodium, potassium, ammonium or substituted ammonium. An example is sodium lauryl ether sulphate (SLES). Preferred alkyl ether sulphate is sodium lauryl ether sulphate having an average degree of ethoxylation of from 0.5 to 3, preferably from 1 to 3, more preferably from 2 to 3.

The anionic cleansing surfactants are typically present in hair care composition of the present invention at a level of from 0.5 to 45%, more preferably from 1.5 to 35% and most preferably from 5 to 20%, based on total weight of the hair care composition and including all ranges subsumed therein.

The composition as per the invention optionally and preferably additionally comprises co-surfactants such as amphoteric and zwitterionic surfactants to provide mildness to the composition. Suitable examples include alkyl amine oxides, alkyl betaines, alkyl amidopropyl betaines, alkyl sulphobetaines (sultaines), alkyl amphoacetates, alkyl amphopropionates, alkyl amidopropyl hydroxysultaines, wherein the alkyl group has from 8 to 19 carbon atoms. Preferably, the co-surfactant is a betaine surfactant. Cocamidopropyl betaine (CAPB) is particularly preferred.

When used, the co-surfactant typically makes up from 0.1 to 15%, more preferably from 0.5 to 8% and most preferably from 0.5 to 4% by weight of the hair care composition, based on total weight of the hair care composition and including all ranges subsumed therein.

Preferably the composition of the invention further comprises a suspending agent. Suitable suspending agents are selected from polyacrylic acids, cross-linked polymers of acrylic acid, copolymers of acrylic acid with a hydrophobic monomer, copolymers of carboxylic acid-containing monomers and acrylic esters, cross-linked copolymers of acrylic acid and acrylate esters, heteropolysaccharide gums and crystalline long chain acyl derivatives. The long chain acyl derivative is desirably selected from ethylene glycol stearate, alkanolamides of fatty acids having from 16 to 22 carbon atoms and mixtures thereof. Ethylene glycol distearate and polyethylene glycol 3 distearate are preferred long chain acyl derivatives, since these impart pearlescence to the composition. Polyacrylic acid is available commercially as Carbopol 420, Carbopol 488 or Carbopol 493. Polymers of acrylic acid cross-linked with a polyfunctional agent may also be used; they are available commercially as Carbopol 910, Carbopol 934, Carbopol 941 and Carbopol 980. An example of a suitable copolymer of a carboxylic acid containing monomer and acrylic acid esters is Carbopol 1342. All Carbopol (trademark) materials are available from Goodrich.

Suitable cross-linked polymers of acrylic acid and acrylate esters are Pemulen TR1 or Pemulen TR2. A suitable heteropolysaccharide gum is xanthan gum, for example that available as Kelzan mu.

Mixtures of any of the above suspending agents may be used. Preferred is a mixture of cross-linked polymer of acrylic acid and crystalline long chain acyl derivative.

The suspending agent is generally present in hair care composition of this invention in an amount of from 0.1 to 10%, more preferably from 0.2 to 6%, and most preferably from 0.3 to 4%, based on total weight of the hair care composition and including all ranges subsumed therein.

Preservatives may also be incorporated into the hair care composition of this invention to protect against the growth of potentially harmful microorganisms. Suitable traditional preservatives in addition to the co-solvent which is included in the composition include alkyl esters of parahydroxybenzoic acid, hydantoin derivatives, propionate salts, and a variety of quaternary ammonium compounds. Illustrative yet non-limiting examples of the types of preservatives that may be used in this invention include, for examples, methyl paraben, butyl paraben, propyl paraben, diazolidinyl urea, sodium dehydroacetate, benzyl alcohol, iodopropynyl butylcarbamate, caprylyl glycol, disodium EDTA or mixtures thereof. Preservatives are preferably employed in amounts ranging from 0.01 to 2% by weight of the hair care composition.

The hair care composition of the present invention may contain other ingredients which are common in the art to enhance physical properties and performances. Suitable ingredients include but are not limited to fragrance, dyes and pigments, pH adjusting agents, pearlescers or opacifiers, viscosity modifiers, thickeners, and natural hair nutrients such as botanicals, fruit extracts, sugar derivatives and amino acids.

The compositions of the invention are primarily intended for topical application to at least a portion of the hair and/or scalp of an individual, either in rinse-off or leave-on compositions, preferably in rinse-off compositions like shampoos.

### Method of Preparing Premix and Hair Care Compositions

This invention relates to a method of preparing a premix comprising the steps of:
(i) dissolving the co-solvent in water to produce a solution; and
(ii) adding piroctone olamine in the solution of step (i) to obtain the premix.

Preferably, the co-solvent is selected from sodium salicylate, sodium benzoate, para aminobenzoic, sodium xylene sulfonate, iso-octanol, hexanediol, phenoxyethanol, carbamide, acetamide, carbamate and mixtures thereof, more preferably the co-solvent is sodium salicylate, sodium benzoate, hexanediol, phenoxyethanol or mixtures thereof, and most preferably the co-solvent is sodium salicylate, phenoxyethanol or mixtures thereof.

The water in step (i) is preferably pre-heated to a temperature ranging from 20 to 80° C., more preferably from 40 to 80° C., and most preferably from 50 to 80° C.

The duration of step (i) is preferably between 1 second to 30 minutes, more preferably from 5 seconds to 10 minutes, and most preferably from 30 seconds to 5 minutes.

This invention also relates to a method of preparing a hair care composition comprising the steps of:
(i) dissolving the co-solvent in water to produce a solution;
(ii) adding piroctone olamine in the solution of step (i) to obtain a premix;
(iii) mixing the premix of step (ii) with other ingredients to form the hair care composition.

Preferably, the co-solvent is selected from sodium salicylate, sodium benzoate, para aminobenzoic, sodium xylene sulfonate, iso-octanol, hexanediol, phenoxyethanol, carbamide, acetamide, carbamate and mixtures thereof, more preferably the co-solvent is sodium salicylate, sodium benzoate, hexanediol, phenoxyethanol or mixtures thereof, and most preferably the co-solvent is sodium salicylate, phenoxyethanol or mixtures thereof.

The water in step (i) is preferably preheated to a temperature ranging from 20 to 80° C., more preferably from 40 to 80° C., and most preferably from 50 to 80° C.

The duration of step (i) is preferably between 1 second to 30 minutes, more preferably from 5 seconds to 10 minutes, and most preferably from 30 seconds to 5 minutes.

The invention is also concerned with the hair care composition obtainable and/or obtained by the method of preparing the hair care composition.

The invention is further concerned with a method of treating and/or preventing dandruff comprising the step of applying the hair care composition onto hair and/or scalp of an individual. The method is preferably non-therapeutic.

The following examples are provided to facilitate an understanding of the present invention. The examples are not provided to limit the scope of the claims.

### EXAMPLES

### Example 1

This example demonstrated co-solvent can affect the dissolution rate of piroctone olamine in water. All ingredients are expressed by amount of the total formulation and as level of active ingredient.

TABLE 1

| | Samples | | | | | |
|---|---|---|---|---|---|---|
| Ingredient/g | 1 | 2 | 3 | 4 | 5 | 6 |
| Sodium salicylate | — | 50 | — | 25 | 50 | 25 |
| Phenoxyethanol | — | — | 50 | 25 | — | 25 |
| Piroctone olamine (Octopirox ®) | 50 | 50 | 50 | 50 | — | — |
| Climbazole | — | — | — | — | 50 | 50 |
| Water | 150 | 150 | 150 | 150 | 150 | 150 |

### Formulation Process

Water was first added into a beaker and stirred with four blade agitator at 500 rpm, followed by adding sodium salicylate and mixing for 1-2 minutes until sodium salicylate was fully dissolved. If the sample comprised phenoxyethanol, phenoxyethanol was then added into the beaker and stirred for 1 minute. After that, piroctone olamine or climbazole was added into the beaker and the mixture was kept stirring. The process was carried out at 25° C. or 60° C. The dissolution of piroctone olamine or climbazole was observed and recorded.

### Results

The dissolution of piroctone olamine or climbazole was observed by naked eye and optical microscope OLYMPUS BX51 under 10× magnification. When the appearance of the mixture was transparent with no particles observed by naked eye and there was no particles or crystals observed under optical microscope, it indicated that piroctone olamine or climbazole was fully dissolved. The results were reported in table 2.

TABLE 2

| Dissolving | Samples | | | | | |
|---|---|---|---|---|---|---|
| time/min | 1 | 2 | 3 | 4 | 5 | 6 |
| 25° C. | NA^{a} | 34 | 42 | 22 | NA^{a} | NA^{a} |
| 60° C. | NA^{a} | 6 | 8 | 4 | NA^{a} | NA^{a} |

^{a}NA means that piroctone olamine or climbazole could not be dissolved.

It can be seen from the results that the addition of co-solvent greatly increased the dissolution rate of piroctone olamine. Sample 4 comprised a combination of sodium salicylate and phenoxyethanol showed the highest piroctone olamine dissolution rate.

### Example 2

This example demonstrated the weight ratio of co-solvent to water can affect the dissolution of piroctone olamine in water. All ingredients are expressed by amount of the total formulation and as level of active ingredient.

TABLE 3

| | Samples | | | | | |
|---|---|---|---|---|---|---|
| Ingredient/g | 7 | 8 | 9 | 10 | 11 | 12 |
| Sodium salicylate | 30 | 30 | 30 | 30 | 30 | 30 |
| Piroctone olamine (Octopirox ®) | 50 | 50 | 50 | 50 | 50 | 50 |
| Water | 1000 | 500 | 200 | 170 | 150 | 90 |

The same process was used to prepare the samples as described in Example 1. The piroctone olamine dissolution results were reported in table 4.

TABLE 4

| Dissolv | | | | | | |
|---|---|---|---|---|---|---|
| -ing | Samples | | | | | |
| Status | 7 | 8 | 9 | 10 | 11 | 12 |
| 35° C. | NA^{a} | dissolved | dissolved | dissolved | dissolved | dissolved |

The results showed that the dissolution of piroctone olamine can be affected by the weight ratio of co-solvent to water. Piroctone olamine was not fully dissolved in sample 7. For samples 8 and 9, piroctone olamine was fully dissolved, but the samples were phase separated. Piroctone olamine in sample 10 was fully dissolved but the sample turned out to be a hazy solution. Samples 11 and 12 showed fully dissolution of piroctone olamine which resulted in a clear and homogeneous solution.

### Example 3

This example demonstrated co-solvent can affect the dissolution rate of piroctone olamine in water. All ingredients are expressed by amount of the total formulation and as level of active ingredient.

TABLE 5

| | Samples | | | |
|---|---|---|---|---|
| Ingredient/g | 13 | 14 | 15 | 16 |
| Sodium Benzoate | — | 50 | — | — |
| Hexanediol | — | — | — | 20 |
| Piroctone olamine (Octopirox ®) | 50 | 50 | 15 | 15 |
| Water | 150 | 150 | 150 | 150 |

The same process was used to prepare the samples as described in Example 1. The piroctone olamine dissolution results were reported in table 6.

TABLE 6

| Dissolving | Samples | | | |
|---|---|---|---|---|
| Status | 13 | 14 | 15 | 16 |
| 60° C. | NA^{a} | dissolved | NA^{a} | dissolved |

^{a}NA means that piroctone olamine could not be dissolved.

It can be seen from the results that the addition of co-solvent greatly increased the dissolution rate of piroctone olamine.

1. A premix comprising:
(a) piroctone olamine;
(b) a co-solvent selected from sodium salicylate, sodium benzoate, para aminobenzoic, sodium xylene sulfonate, iso-octanol, hexanediol, phenoxyethanol and mixtures thereof; and
(c) water;
wherein the weight ratio of co-solvent to water (b:c) ranges from 1:20 to 1:1.

2. The premix according to claim 1, wherein the co-solvent is sodium salicylate, sodium benzoate, hexanediol, phenoxyethanol or mixtures thereof.

3. The premix according to claim 1, wherein the co-solvent is a mixture of sodium salicylate and phenoxyethanol.

4. The premix according to claim 1, wherein the weight ratio of co-solvent to water ranges from 1:10 to 1:1.

5. The premix according to claim 1, wherein the water is present in an amount of from 10% to 90% by weight of the premix.

6. The premix according to claim 1, wherein the co-solvent is present in an amount of from 3 to 70% by weight of the premix.

7. The premix according to claim 1, wherein the piroctone olamine is present in an amount of from 0.01 to 70% by weight of the premix.

8. A hair care composition comprising the premix according to claim 1 wherein the co-solvent is present in an amount of from 0.01 to 4% by weight of the composition; wherein said composition is produced by a process comprising the steps of: (i) dissolving the co-solvent in water to produce a solution; (ii) adding piroctone olamine in the solution of step (i) to obtain a premix; and (iii) mixing the premix of step (ii) with other ingredients to form the hair care composition.

9. The hair care composition according to claim 8, wherein the piroctone olamine is present in an amount of from 0.01 to 10% by weight of the composition.

10. The hair care composition according to claim 8, wherein the co-solvent is present in an amount of from 0.1 to 2% by weight of the composition.

11. The hair care composition according to claim 8, wherein the composition further comprises a cationic polymer.

12. The hair care composition according to claim 11, wherein the cationic guar is guar hydroxypropyltrimonium chloride.

13. A method of preparing a premix according to claim 1 comprising the steps of:
(i) dissolving the co-solvent in water to produce a solution; and
(ii) adding piroctone olamine in the solution of step (i) to obtain the premix.

14. A method of preparing a hair care composition according to claim 8 comprising the steps of:
(i) dissolving the co-solvent in water to produce a solution;
(ii) adding piroctone olamine in the solution of step (i) to obtain a premix; and
(iii) mixing the premix of step (ii) with other ingredients to form the hair care composition.

15. A method of treating and/or preventing dandruff comprising the step of applying the hair care composition according to claim 8 onto hair and/or scalp of an individual.

16. The premix according to claim 2, wherein the co-solvent is sodium salicylate, phenoxyethanol, or mixtures thereof.

17. The premix according to claim 4, wherein the weight ratio of co-solvent to water ranges from 1:5 to 1:3.

18. The premix according to claim 5, wherein the water is present in an amount of from 20 to 80%.

19. The premix according to claim 6, wherein the co-solvent is present in an amount of from 7 to 50% by weight of the premix.

20. The premix according to claim 7, wherein the piroctone olamine is present in an amount of from 0.1 to 50% by weight of the premix.
